(19) United States
(12) Patent Application Publication
(10) Pub. No.: US 2024/0191033 A1
(43) Pub. Date: Jun. 13, 2024

(54) DITHIOACETAL-BASED COVALENT ORGANIC FRAMEWORKS

(71) Applicant: ARIZONA BOARD OF REGENTS ON BEHALF OF ARIZONA STATE UNIVERSITY, Scottsdale, AZ (US)

(72) Inventors: Abhinav Acharya, Scottsdale, AZ (US); Arezoo Esrafili, Scottsdale, AZ (US)

(21) Appl. No.: 18/277,731

(22) PCT Filed: Feb. 18, 2022
(86) PCT No.: PCT/US2022/016984

§ 371 Date: Aug. 17, 2023

Related U.S. Application Data
(60) Provisional application No. 63/150,868, filed on Feb. 18, 2021.

(51) Int. Cl. C08G 75/14; A61K 31/451; A61K 31/795; C08K 5/3432

(52) U.S. Cl. CPC C08G 75/14; A61K 31/451; A61K 31/795; C08K 5/3432; C08G 2230/00

(57) ABSTRACT

Disclosed herein are covalent organic frameworks (COFs) comprising dithioacetal linkages, methods of making such COFs, and methods of using the COFs, e.g., for delivery of gases such as nitric oxide and anti-mycobacterial agents such as isoniazid.

### CROSS-REFERENCE. TO RELATED APPLICATIONS

This application claims priority to and the benefit of U.S. Provisional Patent Application No. 63/150,868, filed on Feb. 18, 2021, which is incorporated herein by reference in its entirety.

### TECHNICAL FIELD

Disclosed herein are covalent organic frameworks (COFs) comprising dithioacetal linkages, methods of making such COFs, and methods of using the COFs, e.g., for delivery of gases such as nitric oxide and anti-mycobacterial agents such as isoniazid.

### BACKGROUND

COFs have been utilized to store gases in the energy field, and given their propensity to bind gases at molecular level makes them excellent candidates for controlled delivery of gases. COFs generally have large pore volumes, and thus afford the ability to load large amounts of drugs and gas releasing materials (see, e.g., Ozdemir et al. Front. Energy Res. (2019) doi:10.3389/fenrg.2019.00077; Furukawa et al J. Am. Chem. Soc. 2009, 131(25):8875-8883; Wu et al. Chinese Chem. Lett. 2017, 28(6):1135-1143), Development of COFs for drug delivery is still in its infancy, although a handful of COFs have been developed and loaded with drugs ibuprofen, 5-fluorouracil, and doxorubicin (see, e.g., Bai et al. Chem. Commun. 2016, 52(22): 4128-4131; Rengaraj et al. ACS Appl. Mater. Inter. 2016, 8(14):8947-8955; Fang et al. J. Am. Chem. Soc. 2015, 137(26):8352-8355). However, these COFs include bonds that are not cleavable in the body, which reduces their potential impact for in vivo applications.

### SUMMARY

Disclosed herein are covalent organic frameworks comprising dithioacetal linkages. In some embodiments, the covalent organic framework comprises the reaction product of an aryl aldehyde and an aryl dithiol, In some embodiments, the aryl aldehyde is an aryl dialdehyde having formula:

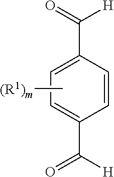

wherein m is 0, 1, or 2, and each R¹ is independently selected from —OH, —SH, —NH₂, —N₃, halo, C₁-C₄ alkyl, C₂-C₄ alkenyl, C₂-C₄ alkynyl, C₁-C₄ alkoxy, and C₁-C₄ haloalkyl, wherein each R¹ can be optionally substituted with a reactive group.

In some embodiments, the aryl aldehyde is an aryl trialdehyde having formula:

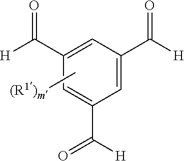

wherein m′ is 0, 1, 2, or 3, and each R^{1′} is independently selected from —OH, —SH, —NH₂, —N₃, halo, C₁-C₄ alkyl, C₂-C₄ alkenyl, C₂-C₄ alkynyl, C₁-C₄ alkoxy, and C₁-C₄ haloalkyl, wherein each R^{1′} can be optionally substituted with a reactive group.

In some embodiments, aryl dithiol has formula:

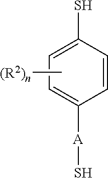

wherein:
n is 0, 1, or 2;
A is a bond or an aryl group substituted with 0, 1, or 2 R² groups; and
each R² is independently selected from —OH, —SH, —NH₂, —N₃, halo, C₁-C₄ alkyl, C₂-C₄ alkenyl, C₂-C₄ alkynyl, C₁-C₄ alkoxy, and C₁-C₄ haloalkyl, wherein each R² can be optionally substituted with a reactive group.

In some embodiments, the covalent organic framework is further functionalized with one or more optionally substituted alkyl or heteroalkyl groups.

In some embodiments, the covalent organic framework comprises the following structure:

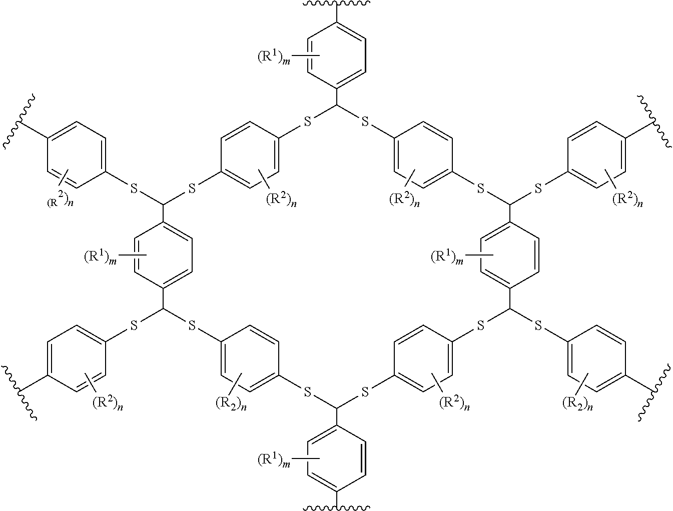

wherein:
each m is 0, 1, or 2;
each n is 0, 1, or 2,
each R¹ and R² is independently selected from —OH, —SH, —S—NO, —NH₂, —N₃, halo, C₁-C₄ alkyl C₂-C₄ alkenyl, C₂-C₄ alkynyl, C₁-C₄ alkoxy, and C₁-C₄ haloalkyl, each of which can be optionally substituted with a reactive group.

In some embodiments, the covalent organic framework comprises the following structure:

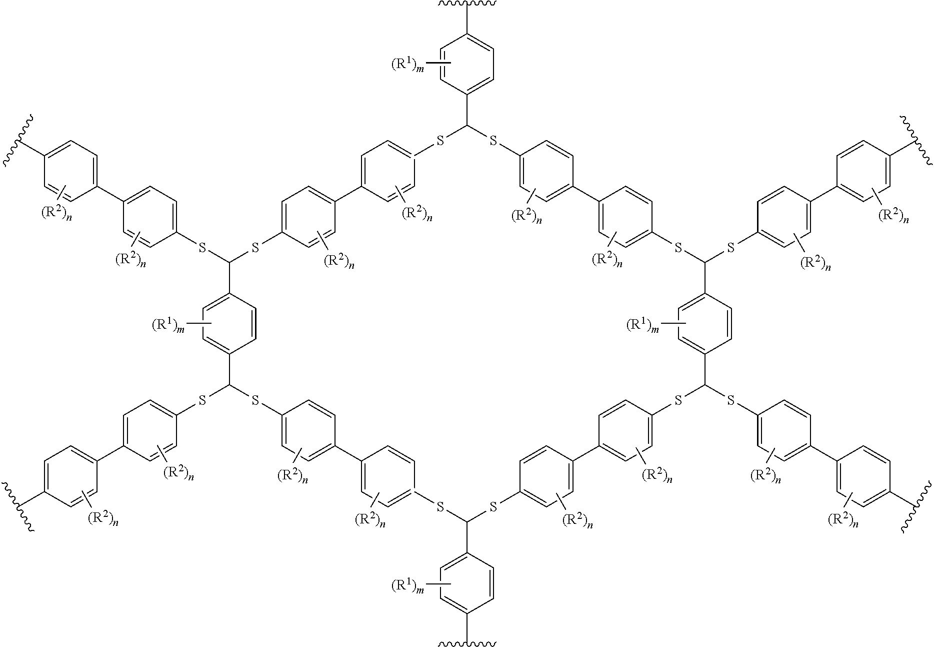

wherein:
each m is 0, 2, 3, or 4;
each n is 0, 1, 2, 3, or 4;
each R¹ and R² is independently selected from —OH, —SH, —S—NO, —NH₂, —N₃, halo, C₁-C₄ allyl C₂-C₄ alkenyl, C₂-C₄ alkynyl, C₁-C₄ alkoxy, and C₁-C₄ haloalkyl, each of which can be optionally substituted with a reactive group.

In some embodiments, the covalent organic framework is further functionalized with one or more polyethylene glycol-containing groups. In some embodiments, the covalent organic framework is loaded with an anti-mycobacterial agent. In some embodiments, the anti-mycobacterial agent is isoniazid. In some embodiments, the covalent organic framework is biodegradable.

Also disclosed herein is a method of synthesizing a covalent organic framework, comprising: reacting an aryl aldehyde with an aryl dithiol. In some embodiments, the aryl aldehyde is an aryl dialdehyde having formula:

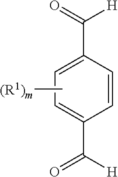

wherein m is 0, 1, 2, 3, or 4, and each R¹ is independently selected from —OH, —SH, —NH₂, —N₃, halo, C₁-C₄ alkyl, C₂-C₄ alkenyl, C₂-C₄ alkynyl, C₁-C₄ alkoxy, and C₁-C₄ haloalkyl, wherein each R¹ can be optionally substituted with a reactive group.

In some embodiments, the aryl aldehyde is an aryl trialdehyde having formula:

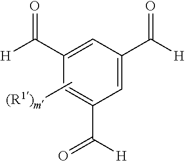

wherein m′ is 0, 1, 2, or 3, and each R^{1′} is independently selected from —OH, —SH, —NH₂, —N₃, halo, C₁-C₄ alkyl, C₂-C₄ alkenyl, C₂-C₄ alkynyl, C₁-C₄ alkoxy, and C₁-C₄ haloalkyl, wherein each R^{1′} can be optionally substituted with a reactive group.

In some embodiments, the aryl dithiol has formula:

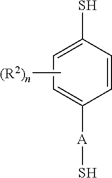

wherein:
n is 0 ; 1., 2, 3, or 4;
A is a bond or an aryl group substituted with 0, 1, or 2 R² groups; and
each R² is independently selected from —OH, —SH, —NH₂, —N₃, halo, C₁-C₄ alkyl, C₂-C₄ alkenyl, C₂-C₄ alkynyl, C₁-C₄ alkoxy, and C₁-C₄ haloalkyl, wherein each R² can be optionally substituted with a reactive group.

Also disclosed herein are particles comprising the covalent organic frameworks.

Also disclosed herein is a method of delivering an anti-mycobacterial agent to a subject in need thereof, comprising administering to the subject an effective amount of a covalent organic framework disclosed herein (e.g., a covalent organic framework loaded with an anti-mycobacterial agent). In some embodiments, the administrating step comprises pulmonary administration. In some embodiments, the subject is infected with a Mycobacterium species. In some embodiments, the covalent organic framework is degraded in the subject after administration and delivery of the anti-mycobacterial agent.

### BRIEF DESCRIPTION OF THE DRAWINGS

FIGS. 1A-1B show powder X-ray diffraction spectra of two COFs disclosed herein: (A) COF-ASU-11; and (B) COF-ASU-12.

FIGS. 2A-2B show scanning electron microscopy images of two COFs disclosed herein at two different magnifications: (A) COF-ASU-11; and (B) COF-ASU-12.

FIG. 3 shows Raman spectroscopy data for a COF disclosed herein (COF-ASU-12) in the presence of acid, hydrogen peroxide, or hydrogen peroxide plus Fenton's reagent.

FIG. 4 shows fluorescence microscopy images of macrophages that had been incubated with two COFs disclosed herein (COF-ASU-11 and COF-ASU-12) that had been loaded with rhodamine, and further stained with DAPI and DID dye.

FIG. 5 shows data demonstrating that two COFs disclosed herein (COF-ASU-11 and COF-ASU-12) can absorb and release isoniazid.

### DETAILED DESCRIPTION

Disclosed herein are dithioacetal-based covalent organic frameworks. These materials are stable in water, acids, and bases, but are labile to reactive oxygen species, such that they can ultimately be degraded in the body by macrophages. These materials are useful for loading drug molecules such as anti-mycobacterial agents (e.g., isoniazid), and for gas-releasing materials such as nitric oxide.

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents, and other references mentioned herein are incorporated by reference in their entirety. The materials, methods, and examples disclosed herein are illustrative only and not intended to be limiting.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Sorrell, Organic Chemistry, 2^{nd} edition, University Science Books, Sausalito, 2006; Smith, March's Advanced Organic Chemistry: Reactions, Mechanism, and Structure, 7^{th} Edition, John Wiley &. Sons, Inc., New York, 2013; Larock, Comprehensive Organic Transformations, 3^{rd} Edition, John Wiley & Sons, Inc., New York. 2018; and Carruthers, Some Modern Methods of Organic Synthesis, 3^{rd} Edition, Cambridge University. Press, Cambridge, 1987; the entire contents of each of which are incorporated herein by reference.

As used herein, the term “alkyl” means a straight or branched saturated hydrocarbon chain containing from 1 to 16 carbon atoms (C₁-C₁₆ alkyl), for example 1 to 14 carbon atoms (C₁-C₁₄ alkyl), 1 to 12 carbon atoms (C₁-C₁₂ alkyl), 1 to 10 carbon atoms (C₁-C₁₀ alkyl), 1 to 8 carbon atoms (C₁-C₈ alkyl), 1 to 6 carbon atoms (C₁-C₆ alkyl), or 1 to 4 carbon atoms (C₁-C₄ alkyl). Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, n-undecyl, and n-dodecyl.

As used herein, the term “alkenyl” refers to a straight or branched hydrocarbon chain containing from 2 to 16 carbon atoms and containing at least one carbon-carbon double bond. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1 -heptenyl, and 3-decenyl.

As used herein, the term “alkynyl” refers to a straight or branched hydrocarbon chain containing from 2 to 16 carbon atoms and containing at least one carbon-carbon triple bond. Representative examples of alkynyl include, but are not limited to, ethynyl, propynyl, and butynyl.

As used herein, the term “alkoxy” refers to an alkyl group, as defined herein, appended to the parent molecular moiety through an oxygen atom. Representative examples of alkoxy include, but are not limited to, methoxy, ethoxy, propoxy, 2-propoxy, butoxy and tert-butoxy.

As used herein, the term “aryl” refers to an aromatic carbocyclic ring system having a single ring (monocyclic) or multiple rings (bicyclic or tricyclic) including fused ring systems, and zero heteroatoms. As used herein, aryl contains 6-20 carbon atoms (C₆-C₂₀ aryl), 6 to 14 ring carbon atoms (C₆-C₁₄ aryl), 6 to 12 ring carbon atoms (C₆-C₁₂ aryl), or 6 to 10 ring carbon atoms (C₆-C₁₀ aryl). Representative examples of aryl groups include, but are not limited to, phenyl, naphthyl, anthracenyl, and phenanthrenyl.

As used herein, the terms “halogen” and “halo” mean F, Cl, Br, or I.

As used herein, the term “haloalkyl” means an alkyl group, as defined herein, in which one or more hydrogen atoms are replaced by a halogen. For example, one, two, three, four, five, six, seven, or eight hydrogen atoms can be replaced by a halogen, or all hydrogen atoms can be replaced by a halogen. Representative examples of haloalkyl include, but are not limited to, fluoromethyl, difluoromethyl, trifluoromethyl, chloromethyl, dichloromethyl, trichloromethyl, 2-fluoroethyl, 2,2-difluoroethyl, 2,2,2-trifluoroethyl, perfluoroethyl, 2-fluoro-2-methylpropyl, 3,3,3-trifluoropropyl, 4-chlorobutyl, 5-chloropentyl, 6-chlorohexyl, 7-chloroheptyl, and 8-chlorooctyl.

As used herein, the term “heteroalkyl” means an alkyl group, as defined herein, in which one or more of the carbon atoms (and any associated hydrogen atoms) are each independently replaced with a heteroatom group such as —NH—, —S—, —S(O)—, —S(O)₂—, and the like. By way of example, 1, 2, or 3 carbon atoms may be independently replaced with the same or different heteroatom group. Examples of heteroalkyl groups include, but are not limited to, —OCH₃, —CH₂OCH₃, —SCH₃, —CH₂SCH₃, —NCH₃, and —CH₂NHCH₃. Heteroalkyl also includes groups in which a carbon atom of the alkyl is oxidized (i.e., is —C(O)—).

As used herein, the term “reactive group” refers to a group that is capable of reacting with another chemical group to form a covalent bond, i.e. is covalently reactive under suitable reaction conditions, and generally represents a point of attachment for another substance. For example, in some embodiments, a reactive group is a carboxylic acid, an isocyanate, an isothiocyanate, a maleimide, an azide, an alkyne, or an ester such as a succinimidyl, pentafluorophenyl or tetrafluorophenyl ester.

As used herein, the term “dithioacetal” refers to a group of formula RCH(SR′)(SR″), wherein R, R′, and R″ are each independently carbon based moieties (e.g., alkyl, aryl, or the like).

### Covalent Organic Frameworks

Disclosed herein are covalent organic frameworks (COFs) comprising dithioacetal linkages. The dithioacetal moieties are labile to reactive oxygen species, and therefore can be degraded in the body (e.g., after delivery of an agent loaded inside the COF).

In some embodiments, the COF comprises the reaction product of an aryl aldehyde (e.g., an dialdehyde or an aryl trialdehyde) and an aryl dithiol. For example, in some embodiments, the aryl aldehyde is an aryl dialdehyde has formula:

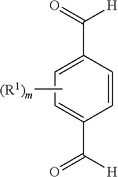

wherein m is 0, 1, 3, or 4, and each R¹ is independently selected from —OH, —SH, —NH₂, —N₃, halo, C₁-C₄ alkyl, C₂-C₄ alkenyl, C₂-C₄ alkynyl, C₁-C₄ alkoxy, and C₁-C₄ haloalkyl, wherein each R¹ can be optionally substituted with a reactive group (e.g., a maleimide, an ester such as an N-hydroxysuccinimidyl ester, or the like). For example, in some embodiments, m is 0 (i.e. the compound is terephthalaldehyde). In some embodiments, m is 2, and each R¹ is hydroxy (e.g., the compound is 2,5-dihydroxyterephthalaldehyde).

In some embodiments, the aryl aldehyde is an aryl trialdehyde having formula:

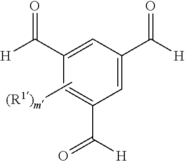

wherein m′ is 0, 1, 2, or 3, and each R^{1′} is independently selected from —OH, —SH, —NH₂, —N₃, halo, C₁-C₄ alkyl, C₂-C₄ alkenyl, C₂-C₄ alkynyl, C₁-C₄ alkoxy, and C₁-C₄ haloalkyl, wherein each R^{1′} can be optionally substituted with a reactive group (e.g., a maleimide, an ester such as an N-hydroxysuccinimidyl ester, or the like). For example, in some embodiments, m is 0 (i.e. the compound is benzene-1,3,5-tricarbaldehyde). in some embodiments, m is 3, and each R¹ is hydroxy (i.e. the compound is 2,4,6-trihydroxybenzene-1,3,5-tricarbaldehyde).

In some embodiments, the aryl dithiol has formula:

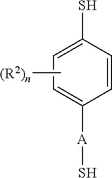

wherein: n is 0, 1, 2, 3, or 4; A is a bond or an aryl group substituted with 0, 1, 2, 3, or 4 R² groups; and each R² is independently selected from —OH, —SH, —NH₂, —N₃, halo, C₁-C₄ alkyl, C₂-C₄ alkenyl, C₂-C₄ alkynyl, C₁-C₄ alkoxy, and C₁-C₄ haloalkyl, wherein each R² can be optionally substituted with a reactive group (e.g., a maleimide, an ester such as an N-hydroxysuccinimidyl ester, or the like). For example, in some embodiments, n is 0 and A is a bond (i.e. the compound is benzene-1,4-dithiol). In some embodiments, n is 0 and A is a phenylene group that is unsubstituted (i.e. the compound is biphenyl-4,4′-dithiol).

In some embodiments, the COF is further functionalized with one or more optionally substituted alkyl groups. For example, when the aryl aldehyde (e.g., aryl dialdehyde or aryl trialdehyde) comprises one or more reactive R¹ groups (e.g., wherein R¹ is —OH) and/or when the aryl dithiol comprises one or more reactive R² groups (e.g., wherein R² is —OH), the COF can be further functionalized with a compound such as 1,4-butanedithiol, ethanolamine, 1,3-propanedithiol, spermidine, or the like. Such moieties covalently attach to the COF via the reactive R¹ and/or R² group(s).

In some embodiments, the COF comprises the following structure:

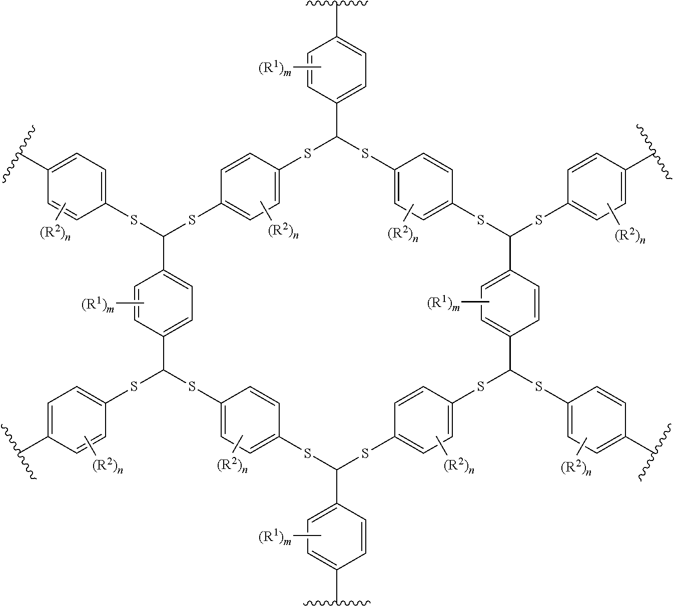

wherein:
each m is 0, 1, or 2;
each n is 0, 1, or 2,
each R¹ and R² is independently selected from —OH, —SH, —S—NO, —NH₂, —N₃, halo, C₁-C₄ alkyl, C₂-C₄ alkenyl, C₂-C₄ alkynyl, C₁-C₄ alkoxy, and C₁-C₄ haloalkyl, each of which can be optionally substituted with a reactive group (e.g., a maleimide, an ester such as an N-hydroxysuccinimidyl ester, or the like).

In some embodiments, the COF comprises the following structure:

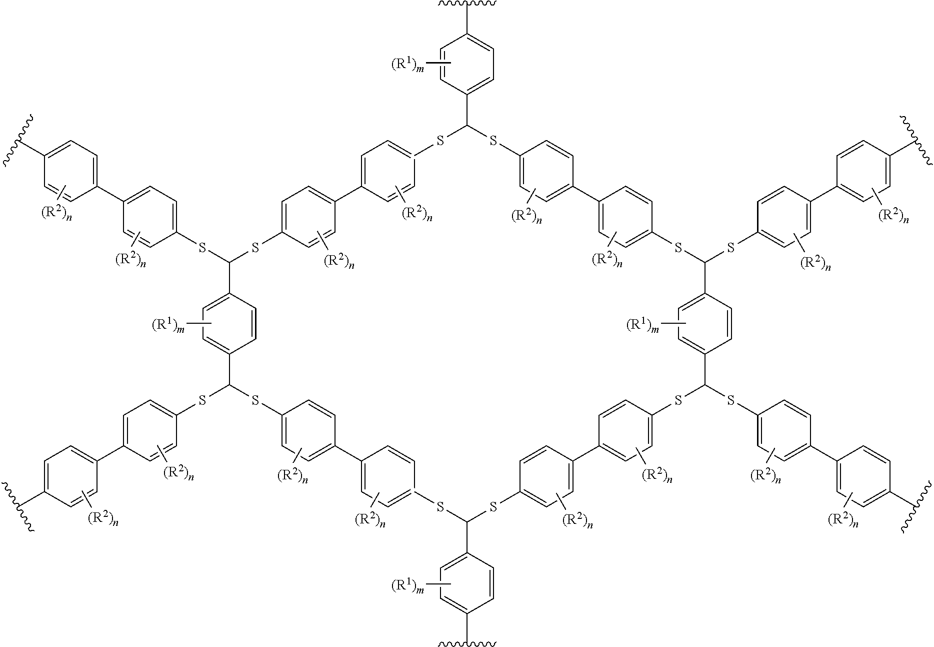

wherein:
each n3 is 0, 1, or 2;
each n is 0, 1, or 2,
each R¹ and R² is independently selected from —OH, —SH, —S—NO, —NH₂, —N₃, halo, C₁-C₁ C₂-C₄ alkenyl, C₂-C₄ alkynyl, C₁-C₄ alkoxy, and C₁-C₄ haloalkyl, each of which can be optionally substituted with a reactive group (e.g., a maleimide, an ester such as an N-hydroxysuccinimidyl ester, or the like).

In the structures shown above, the group

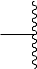

represents either a terminal group or a point of attachment to an additional moiety within the COF. For example, the group

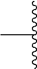

may represent a point of attachment to another dithioacetal moiety, or it may represent a terminal group (e.g., an aldehyde or a thiol). It should be understood that the COFs contain many repeat units derived from the reaction of the aryl aldehyde (e.g., aryl dialdehyde or aryl trialdehyde) with the aryl dithiol, and that the structures shown above illustrate the repeating units. The actual COFs may include many more of such repeat units, as one skilled in the art would appreciate.

In some embodiments, the groups R¹ and R² are directly derived from the starting materials used to prepare the COFs (i.e. the aryl dialdehyde or trialdehyde and the aryl dithiol). In some embodiments, the groups R¹ and R² can be derived from post-COF synthesis reactions. For example, in a COF such as one having a structure shown above in which R¹ and/or R² is SH, the —SH group can be installed by first reacting an —OH functionalized COF with a compound that activates the —OH group (i.e. to install a leaving group such as a mesyl group or a tosyl group), and then reacting that product with sodium sulfide. In a COF such as one having a structure shown above in which R¹ and/or R² is —S—NO, the —S—NO group can be installed by reacting an —SH functionalized COF with gaseous nitric oxide.

In some embodiments, the COF is further functionalized with one or more polyethylene glycol-containing groups. PEGylation can increase the hydrophilicity of the COFs.

In some embodiments, the COF is loaded with an anti-mycobacterial agent, such as isoniazid. The COF can be loaded with the drug, for example, by incubating the COF with the drug for a period of time (e.g., about 6 hours to about 48 hours, e.g., about 24 hours), and then washing the COF to remove un-absorbed compound.

Also disclosed herein is method of synthesizing a covalent organic framework, comprising reacting an aryl aldehyde (e.g., an aryl dialdehyde or an aryl trialdehyde) with an aryl dithiol. In some embodiments, the aryl aldehyde is an aryl dialdehyde having formula:

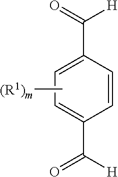

wherein m is 0, 1, or 2, and each R¹ is independently selected from —OH, —SH, —NH₂, —N₃, halo, C₁-C₄ alkyl, C₂-C₄ alkenyl, C₂-C₄ alkynyl, C₁-C₄ alkoxy, and C₁-C₄ haloalkyl, each of which can be optionally substituted with a reactive group (e.g., a maleimide, an ester such as an N-hydroxysuccinimidyl ester, or the like). For example, in some embodiments, m is 0 (i.e, the compound is terephthalaldehyde). In some embodiments, m is 2, and each R¹ is hydroxy (e.g., the compound is 2,5-dihydroxyterephthalaldehyde).

In some embodiments, the aryl aldehyde is an aryl trialdehyde having formula:

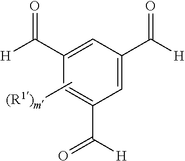

wherein m′ is 0, 1, 2, or 3, and each R^{1′} is independently selected from —OH, —SH, —NH₂, —N₃, halo, C₁-C₄ alkyl, C₂-C₄ alkenyl, C₂-C₄ alkynyl, C₁-C₄ alkoxy, and C₁-C₄ haloalkyl, wherein each R^{1′} can be optionally substituted with a reactive group (e.g., a maleimide, an ester such as an N-hydroxysuccinimidyl ester, or the like). For example, in some embodiments, m is 0 (i.e, the compound is benzene-1,3,5-tricarbaldehyde). In some embodiments, in is 3, and each R¹ is hydroxy (i.e. the compound is 2,4,6-trihydroxybenzene-1,3,5-tricarbaldehyde).

In some embodiments, the aryl dithiol has formula:

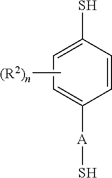

wherein: n is 0, 1, or 2; A is a bond or an aryl group substituted with 0, 1, or 2 R² groups; and each R² is independently selected from —OH, —SH, —NH₂, —N₃, halo, C₁-C₄ alkyl, C₂-C₄ alkenyl, C₂-C₄ alkynyl, C₁-C₄ alkoxy, and C₁-C₄ haloalkyl, wherein each R² can be optionally substituted with a reactive group (e.g., a maleimide, an ester such as an N-hydroxysuccinimidyl ester, or the like). For example, in some embodiments, n is 0 and A is a bond (i.e. the compound is benzene-1,4-dithiol).In some embodiments, n is 0 and A is a phenylene group that is unsubstituted (i.e. the compound is biphenyl-4,4′-dithiol).

In some embodiments, the aryl aldehyde (e.g., aryl dialdehyde or aryl dialdehyde) and the aryl dithiol are reacted in an organic solvent, such as ethyl acetate. In some embodiments, a second solvent is added, such as N,N-dimethylformamide (DMF). In some embodiments, the aryl aldehyde (e.g., aryl dialdehyde or aryl dialdehyde) and the aryl dithiol are reacted in the presence of an acid, such as hydrochloric acid.

To synthesize the COF, the aryl aldehyde (e.g., aryl dialdehyde or trialdehyde) and the aryl dithiol can be reacted (e.g., in an organic solvent) for about 6 hours to about 96 hours, e.g., about 6 hours, about 12 hours, about 18 hours, about 24 hours, about 30 hours, about 36 hours, about 42 hours, about 48 hours, about 54 hours, about 60 hours, about 66 hours, about 72 hours, about 78 hours, about 84 hours, about 90 hours, or about 96 hours. In some embodiments, the aryl dialdehyde and the aryl dithiol are reacted (e.g., in an organic solvent) for about 72 hours. In some embodiments, the aryl dialdehyde and the aryl dithiol can be reacted (e.g., in an organic solvent) at ambient temperature (i.e. about 20-25° C.), or at a reduced temperature (e.g., about 15° C., about 10° C., about 5° C., about 0° C., about −5° C., about −10 ° C., about −15° C., about −20° C., about −25° C., about −30° C., about −35° C., or about −40° C.). In some embodiments, the aryl dialdehyde and the aryl dithiol can be reacted (e.g., in an organic solvent) at ambient temperature. In some embodiments, the aryl dialdehyde and the aryl dithiol can be reacted (e.g., in an organic solvent) at about −20° C.

The prepared COFs can be isolated and purified by methods well-known to those skilled in the art. Examples of conventional methods for isolating and purifying compounds can include, but are not limited to, chromatography on solid supports such as silica gel, alumina, or silica derivatized with alkylsitane groups, by recrystallization at high or low temperature with an optional pretreatment with activated carbon, thin-layer chromatography, distillation at various pressures, sublimation under vacuum, and trituration, as described for instance in “Vogel's Textbook of Practical Organic Chemistry,” 5th edition (1989), by Furniss, Hannaford, Smith, and Tatchell, pub. Longman Scientific &. Technical, Essex CM20 2JE, England. In some embodiments, the COFs crystallize directly from the reaction mixture and can be isolated, e.g., by filtration or centrifugation. In some embodiments, the COFs can be dried, e.g., by air-drying.

Reaction conditions and reaction times for each individual step can vary depending on the particular reactants employed. Unless otherwise described, the starting materials and reagents are either commercially available or can be prepared by one skilled in the art from commercially available materials using methods described in the chemical literature.

Standard experimentation, including appropriate manipulation of the reaction conditions, reagents and sequence of the synthetic route, protection of any chemical functionality that cannot be compatible with the reaction conditions, and deprotection at a suitable point in the reaction sequence of the method are included in the scope of the disclosure. Suitable protecting groups and the methods for protecting and deprotecting different substituents using such suitable protecting groups are well known to those skilled in the art; examples of which can be found in PGM Wuts and TW Greene, in Greene's book titled Protective Groups in Organic Synthesis (4^{th} ed.), John Wiley & Sons, NY (2006).

The synthetic schemes and specific examples as described are illustrative and are not to be read as limiting the scope of the disclosure or the claims. Alternatives, modifications, and equivalents of the synthetic methods and specific examples are contemplated.

In some embodiments, the COFs can be formed as particles. For example, particles can he formed using an emulsion method, such as an oil-in-water emulsion or a water-in-oil emulsion. For example, the aryl aldehyde and aryl dithiol can be dissolved in an oil phase (e.g., dichloromethane, ethyl acetate, chloroform, or other oils), to which can be added a water phase that can include any catalysts (e.g., acetic acid, hydrofluoric acid, Scare based catalysts, or others). The emulsion can be generated by combining the oil phase and the water phase. The emulsion composition can further include one or more emulsifying agents, such as naturally occurring detergents, esters or partial esters derived from combinations of fatty acids, water soluble emulsifiers such as Tween 80, Tween 20 and others, or oil-soluble emulsifiers such as Span 80 and others. The emulsion can be generated using an industrial homogenizer, a sonicator, stirring, or any other methods. The reaction will take place at the water-oil interface. In some embodiments, the particles can have an average diameter of about 1 nanometer to about 100 micrometer.

### Methods of Use

The disclosed COFs can be used in a variety of methods. For example, the COFs can be used for nitric oxide delivery, or delivery of anti-mycobacterial drugs such as isoniazid.

Gases such as nitric oxide are used by the immune system to modulate immune responses in wound repair, infection treatment and tissue engineering. Delivery of NO has accordingly been sought after for modulating immune responses. As described herein, COFs can be loaded with NO by incubating an appropriate functionalized COF with NO (e.g., under pressure). The NO-loaded COFs can then he used in methods of delivering nitric oxide, where the NO is released from the COF in a sustained manner. Such delivery can be used, for example, to aid in treatment of a Mycobacterium infection, such as an M. avium infection, as bolus NO has been demonstrated to eliminate M. avium infection (see, e.g., González-Pérez et al. Infect Immun. 2013, 81(11):4001-12; Baldwin et al. PLoS Negl. Trop. Dis. 2019, 1(2):e000708). Sustained delivery of NO may also find use in many other applications, such as the treatment of cardiovascular disease states.

As demonstrated herein, the disclosed COFs are capable of absorbing and releasing isoniazid, an important antibiotic used for treatment of tuberculosis and for treatment of non-tuberculosis mycobacteria, such as Mycobacterium avium. Notably, soluble isoniazid is not very effective against M. avium due to difficulty in penetrating the lipid membrane and low intracellular concentration (see, e.g., Mdluli et al. Mol. Microbiol. 1998, 27(6):1223-1233). However, the COFs are expected to be able to deliver a large amount of isoniazid intracellularly, increasing the ability to effectively kill M. avium.

COFs disclosed herein can also be simultaneously loaded with NO and isoniazid, and used in a method of delivering both NO and isoniazid in a sustained manner.

in some embodiments, disclosed herein is a method of delivering an anti-mycobacterial agent to a subject in need thereof, comprising administering to the subject an effective amount of a COF disclosed herein (e.g., a COF loaded with an anti-mycobacterial agent such as isoniazid).

As the COFs disclosed herein are biodegradable, the COF is degraded in the subject after administration and delivery of the anti-mycobacterial agent. This imparts a significant advantage to the disclosed COFs, as they can be cleared from the subject's system after delivery of the active compound.

i. Pharmaceutical Compositions

For use in methods described herein, the disclosed COFs may be incorporated into pharmaceutical compositions suitable for administration to a subject (such as a patient, which may be a human or non-human). The pharmaceutical compositions may include a “therapeutically effective amount” or a “prophylactically effective amount” of the COF. A “therapeutically effective amount” refers to an amount effective, at dosages and for periods of time necessary, to achieve the desired therapeutic result. A therapeutically effective amount of the composition may be determined by a person skilled in the art and may vary according to factors such as the disease state, age, sex, and weight of the individual, and the ability of the composition to elicit a desired response in the individual. A therapeutically effective amount is also one in which any toxic or detrimental effects of a compound of the disclosure are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease or condition, the prophylactically effective amount will be less than the therapeutically effective amount.

The pharmaceutical compositions may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator,

Thus, the compounds and their pharmaceutically acceptable salts may be formulated for administration by, for example, solid dosing, eye drop, in a topical oil-based formulation, injection, inhalation (either through the mouth or the nose), implants, or oral, buccal, parenteral, or rectal administration. Techniques and formulations may generally be found in “Remington's Pharmaceutical Sciences,” (Meade Publishing Co., Easton, Pa.). Therapeutic compositions must typically be sterile and stable under the conditions of manufacture and storage.

In some embodiments, the compounds and their pharmaceutically acceptable salts may be formulated for administration by inhalation. Methods of administration of pharmaceuticals and other substances by inhalation are well-known. In general, compounds delivered as aerosols have a particle range of about 0.5 to about 6 μm. Methods known in the art to generate and deliver such aerosols include nebulizers (liquid formulations), dry powder inhalers (dry powder formulations), and metered dose inhalers (drug formulation suspended in a propellant that evaporates virtually instantaneously). Such delivery methods are well-known in the art. See, e.g., M. Keller (1999) Int. J. Pharmaceutics 186:81-90; M. Everard (2001) J. Aerosol Med. 14 (Suppl 1): S-59-S-64; Togger and Brenner (2001) Am. J. Nursing 101:26-32. Commercially available aerosolizers for liquid formulations, including jet nebulizers and ultrasonic nebulizers, are useful in the methods of the invention. For delivery in liquid form, liquid formulations can be directly aerosolized and lyophilized powder can be aerosolized after reconstitution. For delivery in dry powder form, the formulation may be prepared as a lyophilized and milled powder. In additions, formulations may be delivered using a fluorocarbon formulation or other propellant and a metered dose dispenser. For delivery devices and methods, see, e.g., U.S. Pat. Nos. 4,137,914; 4,174,712; 4,524,769; 4,667,688; 5,672,581; 5,709,202; 5,780,014; 5,672,581; 5,915,378; 5,997,848; 6,123,068; 6,123,936; 6,397,838.

ii. Dosages

It will be appreciated that appropriate dosages of the COFs, and compositions comprising the COFs, can vary from patient to patient. Determining the optimal dosage will generally involve the balancing of the level of therapeutic benefit against any risk or deleterious side effects of the treatments described herein. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g. in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

iii. Combination Therapies

A COF described herein may be used in combination with other known therapies. Administered “in combination,” as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery.” in other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

A compound or composition described herein and the at least one additional therapeutic agent can be administered simultaneously, in the same or in separate compositions, or sequentially. For sequential administration, the compound described herein can be administered first, and the additional agent can be administered subsequently, or the order of administration can be reversed.

The COFs of the disclosure can also be used in combination with other drugs. For example, the COFs can be used in combination with other known drugs for treating the disorder of interest (e.g., a Mycobacterium infection).

The following examples further illustrate aspects of the disclosure beat, of course, should not be construed as in any way limiting its scope.

### EXAMPLES

### Example 1: COF Syntheses

COF-ASU-11—Method 1. 100 mg benzene-1,4-dithiol was dissolved in 100 mL ethyl acetate (EtOAc). 44.93 mg terephthalaldehyde was dissolved in 50 mL EtOAc. These solutions were divided equally into ten vials (20 mL capacity). 500 μL is N,N-dimethylformamide (DMF) was added to each vial. Next, 500 μL 1N HCl was added to each vial. The vials were tightly capped and held at room temperature for 72. hours. After 72 hours, the contents of each vial were isolated and washed with 20 mL of DMF (2×10 mL), then with 20 mL EtOAc (2×10 mL). The COFs thus generated were air dried, with yields of 90%.

COF-ASV-11—Method 2. 100 mg benzene-1,4-dithiol was dissolved in 50 ml. EtOAc, and this solution was divided equally into five vials, which were cooled to −20° C. 45 mg terephthalaldehyde was dissolved in 50 mL EtOAc, and cooled to −20° C. Slowly, 1 mL at a time, the 10 mL terephthalaldehyde solution was added to the 10 mL benzene-1,4-dithiol solution, maintaining the temperature at −20° C. The vials were tightly capped and held at 4° C. for 72 hours. The contents of the vials were then transferred to 50 mL falcon tubes and centrifuged at 2000×g for 10 minutes. The resulting solids were washed with EtOAc (3×50 mL), and then air dried, with yields of 90%, and solid-state NMR showed formation of new peaks, suggesting that the reaction moved forward.

COF-ASU-12—Method 1. 100 mg biphenyl-4,4′-dithiol was dissolved in 100 mL EtOAc. 29.27 mg terephthalaldehyde was dissolved in 50 mL of EtOAc. These solutions were divided equally into ten vials (20 mL capacity). 500 μL DMF was added to each vial. Next, 500 μL 1N HCl was added to each vial. The vials were tightly capped and held at room temperature for 72 hours. After 72 hours, the contents of each vial were isolated and washed with 20 mL of DMF (2×10 μL), then with 20 mL EtOAc (2×10 mL). The COFs thus generated were air dried with 90% yields.

COF-ASU-12—Method 1. 100 mg biphenyl-4,4′-dithiol was dissolved in 50 mL EtOAc, and this solution was divided equally into five vials, which were cooled to −20° C. 40 mg terephthalaldehyde was dissolved in 50 mL EtOAc, and cooled to −20° C. Slowly, 1 mL at a time, the 10 mL terephthalaldehyde solution was added to the 10 mL biphenyl-4,4′-dithiol, maintaining the temperature at −20° C. The vials were tightly capped and held at 4° C. for 72 hours. The contents of the vials were then transferred to 50 mL falcon tubes and centrifuged at 2000×g for 10 minutes. The resulting solids were washed with EtOAc (3×50 mL), with 90% yield and solid-state NMR showed formation of new peaks, suggesting that the reaction moved forward

COF-ASU-13. This compound was synthesized in a manner analogous to that described above for COF-ASU-11—Method 1, using 100 mg of benzene-1,4-dithiol and 46.21 mg of 2,4,6-trihydroxybenzene-1,3,5-tricarbaldehyde. Yield: 26.5 mg.

COF-ASU-14. This compound was synthesized in a manner analogous to that described above for COF-ASU-11—Method 1, using 100 mg of benzene-1,4-dithiol and 35.651 mg of benzene 1,3,5-tricarboxaldehyde. Yield: 90.8 mg.

COF-ASU-45. This compound was synthesized in a manner analogous to that described above for COF-ASU-11—Method 1, using 100 mg of dibenzene-4,4′-dithiol and 30.103 mg of 2,4,6-trihydroxybenzene-1,3,5-tricarbaldehyde. Yield: 126.6 mg.

COF-ASU-16. This compound was synthesized in a manner analogous to that described above for COF-ASU-11—Method 1, using 100 mg of dibenzene-4,4′-dithiol and 23.22 mg of benzene 1,3,5-tricarboxaldehyde. Yield: 63.5 mg.

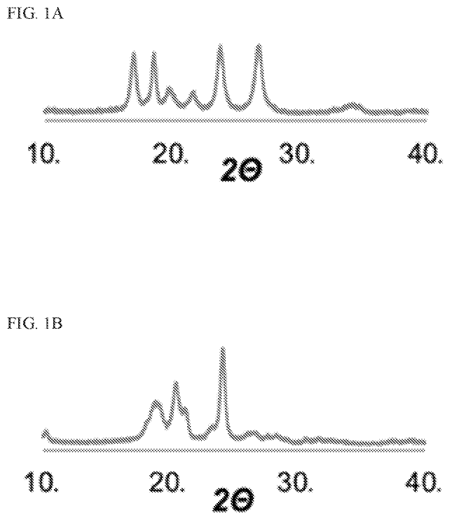

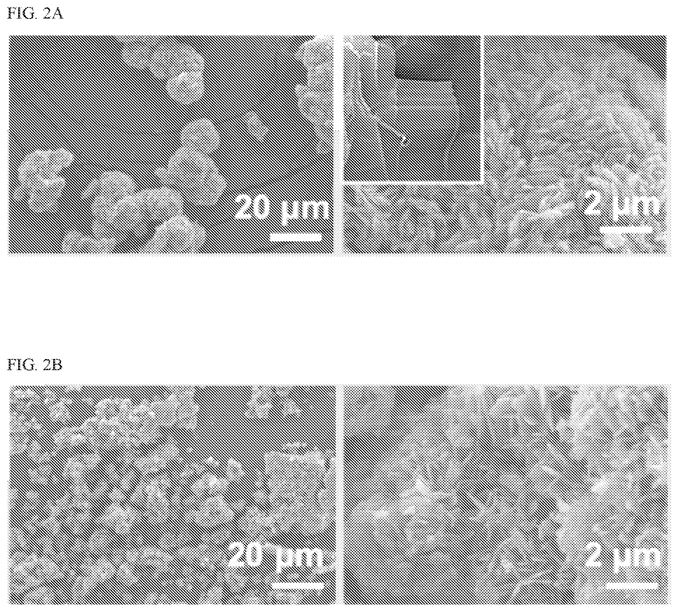

Additional Characterization Data, COF-ASU-11 and COF-ASU-12 were additionally characterized by powder X-ray diffraction (pXRD) and by scanning electron microscopy (SEM). The pXRD spectra are shown in FIGS. 1A-1B, and demonstrate that the COFs are crystalline in nature. The SEM images are shown in FIGS. 2A-2B, and show that the COEs form as two-dimensional sheets and are layered on top of each other.

### Example 2: Degradation Experiments

Calcium peroxide degradation. 0 mg, 1 mg, 5 mg, and 10 mg calcium peroxide will be weighed out into each of four separate Eppendorf tubes. 5 mg of a COF will be added to each tube, then 1 ml, water will be added to each tube. The tubes will be incubated for 2 hours, then centrifuged at 5000×g for 5 minutes, and the pellet will be washed with 3×1 mL DMF, then 3×1 mL deionized water, and then 3×1 mL ethanol. The final pellet will be resuspended in 1 mL ethanol and transferred to a vial and dried at 37° C. for about 24 hours. The product will be then weighed and the weight loss recorded.

Hydrogen peroxide and HO degradation. 5 mg of a COF was placed into each of four reaction tubes, to which was added either 10 mL water, 10 nit of a 1M H₂O₂ solution, 10 mL of 1N HCl solution, or 10 mL of a 1M H₂O₂ solution+0.01 mg iron(II) chloride (Fenton's reagent). The tubes were closed and incubated for 2 hours., then centrifuged at 2000×g for 5 minutes, and the pellet was washed with 3×10 mL DMF, then 3×10 mL deionized water, then 3×10 mL ethanol. The final pellet was dried at 37° C. for about 24 hours.

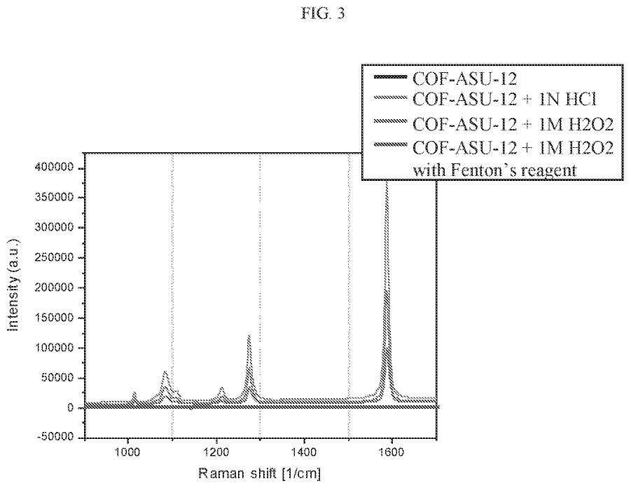

Raman spectroscopy was performed on the resulting materials, and are shown in FIG. 3. The characteristic dithioacetal peak observed at 1585 cm⁻¹ remained for samples of COFs incubated with deionized water, 1M H₂O₂, and 1N HCl, whereas this peak disappeared when the COF was incubated in with H₂O₂ and Fenton's reagent. These data suggest that the crystal structure of the COFs is stable in the reagents that can be found in the body (e.g., acid in the stomach and intracellular H₂O₂ in immune cells) but degrade in the presence of oxygen radicals.

### Example 3: Macrophage Association

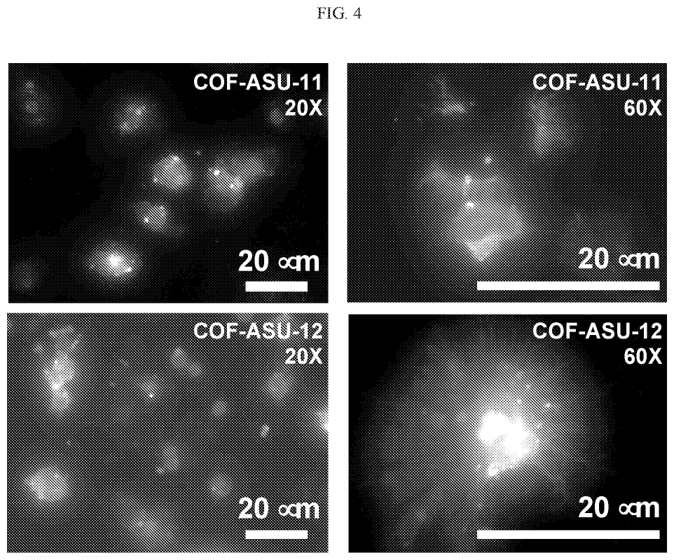

To test whether macrophages can associate with COFs, COF-ASU-11 and COF-ASU-12 were incubated with rhodamine, and then incubated with RAW 264.7 macrophages for 24 hours. These cells were then stained with DAPI for visualizing nucleus and DID dye for visualizing the membrane of these cells. Images were obtained using fluorescent microscope, which shows that the macrophages were able to associate with the COFs (FIG. 4). This suggests that COFs loaded with NO and/or isoniazid will be able to deliver either NO and/or isoniazid to macrophages that may or may not be infected with a Mycobacterium species.

### Example 4: Isoniazid Absorption and Release

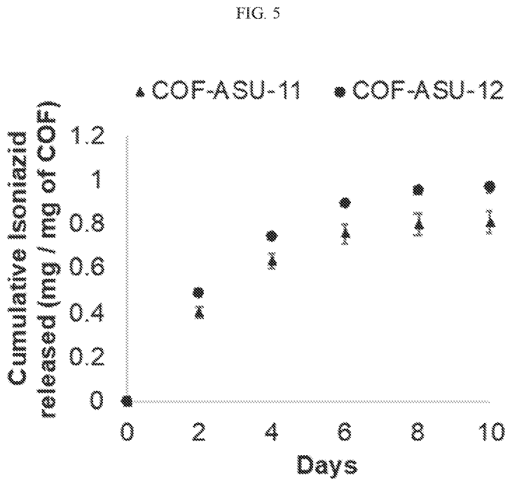

To test whether COFs are able to absorb isoniazid and release them in a sustained manner, isoniazid was incubated with COF-ASU-11 and COF-ASU-12 for 24 hours, and then washed to remove unabsorbed isoniazid. Next, the COFs were incubated in phosphate buffered saline (PBS) and the supernatant was removed every 48 hours and replenished with fresh PBS. HPLC was utilized to quantify isoniazid released from COFs in PBS. COF-ASU-12 was able to absorb and release isoniazid more than COF-ASU-11 (FIG. 5), potentially due to larger pore size. These data suggest that dithioacetal-based COFs were able to absorb isoniazid and release them in a sustained manner for 10 days.

### Example 5: Biocompatibility and Delivery Aspects

Different amounts of COFs (1 mg, 5 mg and 10 mg) will be incubated in simulated lung fluid (SLF—commercially purchased) for 2 hrs, 8 hrs, 24 hrs and 48 hrs, and the weight loss of these COFs will be determined after washing SLF with DI H₂O and lyophilizing the remaining water. Moreover, Raman spectroscopy will be performed on the remaining COFs, which will provide insights into the change in structure of the crystals after incubation in SLF.

To test biocompatibility of COFs, alveolar macrophages and A549 lung epithelial cell lines will be purchased from commercial sources, cultured in appropriate media and used before passage 5. The cells will be seeded in tissue culture plates and treated with COFs at different concentrations (0.001 mg/mL to 0.1 mg/mL) for 24 hours, 48 hours or 72 hours. The cell viability will be determined using an MTT assay (which identifies metabolic activity) and analysis via plate reader, apoptosis will be determined by staining with Annexin V (identifies cell membrane inversion) and analysis via flow cytometry, whereas total dead cells will be determined by staining with live/dead ef780 dye (identifies ruptured cells) and analysis via flow cytometry.

For additional release studies. COFs will be loaded with isoniazid and release in cell culture media and simulated lung fluid will be performed. Moreover, COFs will be incubated with alveolar macrophages for 2 hours and then washed away. The amount of isoniazid present intracellularly will then be isolated by lysing the cells, and the amount of isoniazid will be determined using HPLC.

To test if the COFs are able to kill M. avium different concentrations of COFs (0.001 mg/mL to 1 mg/mL) will be incubated with M. avium 7 H9 bacteria culture media. The number of bacteria surviving after 24 hours of treatment will be determined by plating the cells in 7H11 agar plates.

To test if COFs can kill intracellular M. avium, alveolar macrophages will be infected with multiplicity of infection (MOI) of 1 (1:1 macrophage:M. avium) and incubated for 3 days. After 3 days, and induction of infection, these macrophages will be treated with COFs (0.01 mg/ml and 0.1 mg/mL) for 2 hours. The COFs will be then washed away, and macrophages will be cultured for another 7 days. Next, macrophages will be lysed, and the lysate will be plated on 7H11 agar plate for counting the colony forming units (CFU).

### Example 6: NO loading and Modulation of Macrophage Function

COFs having appropriate functional groups (e.g., thiolated COFs) will be loaded with NO by incubating them with NO under pressure for 72 hours. To test the loading capacity of NO per gram of COFs, NO-loaded COFs will be heated to 37° C. in pH 5 buffer (acid catalyzes release of NO) and a Griess Assay and absorbance spectrophotometer (plate reader) will be utilized to measure the amount of NO. To test the release kinetics, NO-loaded COFs will be incubated in pH 5 buffer (chosen since endosomes are at this pH inside macrophages) at 37° C. and the buffer will be replaced every 24 hours with fresh buffer. The amount of NO generated in the supernatant will be determined using the Griess Assay as well.

Next, the effectiveness of NO-loaded COFs to kill M. avium will be tested. Briefly, the NO-loaded COFs will be incubated with Al avium infected macrophages for 2. hours. The COFs will be washed away and the macrophages will be cultured for another 7 days (doubling rate of M. avium˜24 hours). After 7 days of culture, macrophages will be lysed and the number of M. avium will be determined using 7H11 agar plates. Lastly, macrophages will be cultured with NO-loaded COFs, and the ability to get activated be tested by culturing these cells in the presence of lipopolysaccharide and NO-loaded. COFs. These macrophages will then be stained with TN-Fα, IL-10, MHC-II, CD80 and CD86 antibodies, and the expression will be determined via flow cytometry.

### Example 7: Loading and Release of NO and Isoniazid Simultaneously

COFs will be simultaneously loaded with NO and isoniazid as discussed above. The ability of these drug-loaded COFs to release isoniazid and NO simultaneously will be tested in pH 5 buffer at 37° C. The amount of isoniazid released will be determined using HPLC, and the amount of NO released will be determined using Griess Assay and absorbance spectrophotometer.

The ability of these COFs to kill extracellular M. avium will be tested by incubating 0 mg/mL to 1 mg/mL of these COFs with 10⁶ CFU of M. avium for 3 days in 7H9 culture broth. The number M. avium surviving will be determined by plating these cells on 7H11 agar plates and counting the colonies.

The ability of multi-drug loaded COFs to kill intracellular M. avium will be tested as well under this objective. In this objective the alveolar macrophages will be infected with MOI of 1. These macrophages will then be incubated with drug loaded COFs for 2 hours and the COFs will be washed off, The controls will include COFs without any drugs, COFs loaded with NO only, COFs loaded with isoniazid only, no COF control and positive control of Rifampin (an antibiotic known to kill M. avium at high dosages). The macrophages will be cultured for another 7 days, lysed and the amount of M. avium will be determined by counting colonies on 7H11 agar plates.

Lastly, the ability of COFs to induce macrophages to generate cytokine responses of TNFα, IFNα, IFNγ and IL-10 will be determined using enzyme linked immunosorbent assays. Moreover, macrophage activation after COF treatment will be determined by assessing the surface expression of CD86 and CD80 using flow cytometry.

### Clauses

For reasons of completeness, various aspects of the disclosure are set forth in the following numbered clauses.

Clause 1. A covalent organic framework comprising dithioacetal linkages.

Clause 2. The covalent organic framework of Clause 1, wherein the covalent organic framework comprises the reaction product of an aryl aldehyde and an aryl dithiol.

Clause 3. The covalent organic framework of Clause 2, wherein the aryl aldehyde is an aryl dialdehyde having formula:

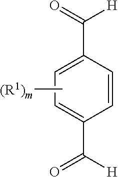

wherein m is 0, 1, or 2. and each R¹ is independently selected from —OH, —SH, —NH₂, —N₃, halo, C₁-C₄ alkyl, C₂-C₄ alkenyl, C₂-C₄ alkynyl, C₁-C₄ alkoxy, and C₁-C₄ haloalkyl, wherein each R¹ can be optionally substituted with a reactive group.

Clause 4. The covalent organic framework of Clause 2, wherein the aryl aldehyde is an aryl trialdehyde having formula:

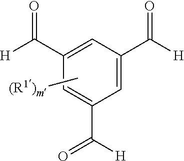

wherein m′ is 0, 1, 2, or 3, and each R^{1′} is independently selected from —OH, —SH, —NH₂, —N₃, halo, C₁-C₄ alkyl, C₂-C₄alkenyl, C₂-C₄ alkynyl, C₁-C₄alkoxy, and C₁-C₄ haloalkyl, wherein each R^{1′} can be optionally substituted with a reactive group.

Clause 5. The covalent organic framework of Clause 2 or Clause 3, wherein the aryl dithiol has formula:

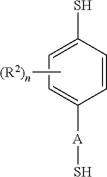

wherein:
n is 0, 1, or 2;
A is a bond or an aryl group substituted with 0, 1, or 2 R² groups; and
each R² is independently selected from —OH, —SH, —NH₂, —N₃, halo, C₁-C₄ alkyl, C₂-C₄ alkenyl, C₂-C₄ alkynyl, C₁-C₄ alkoxy, and C₁-C₄ haloalkyl, wherein each R² can be optionally substituted with a reactive group.

Clause 6. The covalent organic framework of any one of Clauses 1-5, wherein the covalent organic framework is further functionalized with one or more optionally substituted alkyl or heteroalkyl groups.

Clause 7. The covalent organic framework of any one of Clauses 1-5, comprising the following structure:

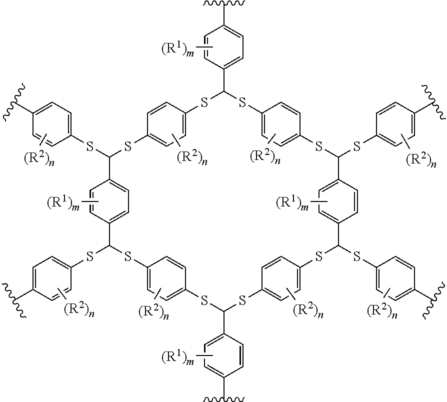

wherein:
each m is 0, 1, or 2;
each n is 0, 1, or 2,
each R¹ and R² is independently selected from —OH, —SH, —S—NO, —NH₂, —N₃, halo, C₁-C₄ alkyl, C₂-C₄ alkenyl, C₇-C₄ alkynyl, C₁-C₄ alkoxy, and C₁-C₄ haloalkyl, each of which can be optionally substituted with a reactive group.

Clause 8. The covalent organic framework of any one of Clauses 1-5, comprising the following structure:

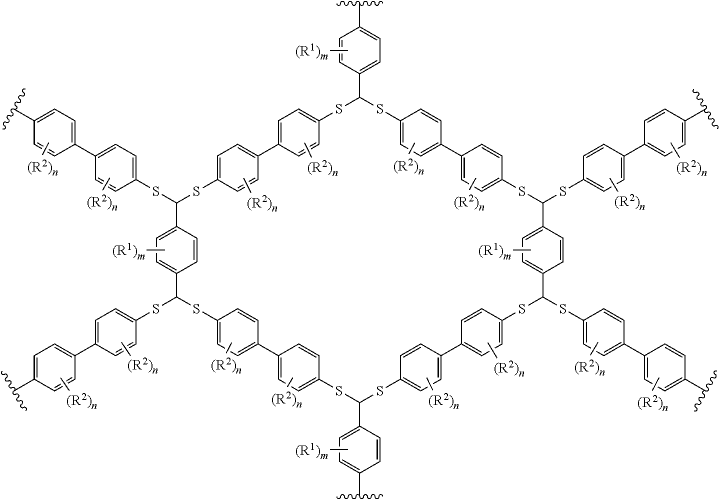

wherein:
each m is 0, 1, 2, 3, or 4;
each n is 0, 1, 2, 3, or 4;
each R¹ and R² is independently selected from —OH, —SH, —S—NO, —NH₂, —N₃, halo, C₁-C₄ alkyl, C₂-C₄ alkenyl, C₂-C₄ alkynyl, C₁-C₄ alkoxy, and C₁-C₄ haloalkyl, each of which can be optionally substituted with a reactive group.

Clause 9. The covalent organic framework of any one of Clauses 1-8, wherein the covalent organic framework is further functionalized with one or more polyethylene glycol-containing groups.

Clause 10. The covalent organic framework of any one of Clauses 1-8, wherein the covalent organic framework is loaded with an anti-mycobacterial agent.

Clause 11. The covalent organic framework of Clause 10, wherein the anti-mycobacterial agent is isoniazid.

Clause 12. The covalent organic framework of any one of Clauses 1-11, wherein the covalent organic framework is biodegradable.

Clause 13. A particle comprising the covalent organic framework of any one of Clauses 1-12.

Clause 14. A method of synthesizing a covalent organic framework, comprising: reacting an aryl aldehyde with an aryl dithiol.

Clause 15. The method of Clause 14, wherein the aryl aldehyde is an aryl dialdehyde having formula:

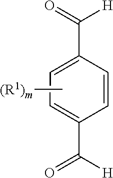

wherein m is 0, 1, 2, 3, or 4, and each R¹ is independently selected from —OH, —SH, —NH₂, —N₃, halo, C₁-C₄ alkyl, C₂-C₄ alkenyl, C₂-C₄ alkynyl, C₁-C₄ alkoxy, and C₁-C₄ haloalkyl, wherein R¹ can be optionally substituted with a reactive group.

Clause 16. The method of Clause 14, wherein the aryl aldehyde is an aryl trialdehyde having formula:

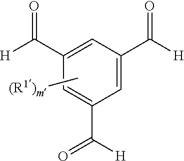

wherein m′ is 0, 1, 2, or 3, and each R^{1′} is independently selected from —OH, —SH, —NH₂, —N₃, halo, C₁-C₄ alkyl, C₂-C₄ alkenyl, C₂-C₄ alkynyl, C₁-C₄ alkoxy, and C₁-C₄ haloalkyl, wherein R^{1′} can be optionally substituted with a reactive group.

Clause 17. The method of any one of Clauses 14-16, wherein the aryl dithiol has formula:

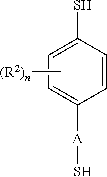

wherein:
n is 0, 1, 2, 3, or 4;
A is a bond or an aryl group substituted with 0, 1, or 2 R² groups; and
each R² is independently selected from —OH, —SH, —NH₂, —N₃, halo, C₁-C₄ alkyl, C₂-C₄ alkenyl, C₂-C₄ alkynyl, C₁-C₄ alkoxy, and C₁-C₄ haloalkyl, wherein R² can be optionally substituted with a reactive group.

Clause 18. A method of delivering an anti-mycobacterial agent to a subject in need thereof, comprising administering to the subject an effective amount of a covalent organic framework of Clause 10 or Clause 11.

Clause 19. The method of Clause 18, wherein the administrating step comprises pulmonary administration.

Clause 20. The method of Clause 18 or Clause 19, wherein the subject is infected with a Mycobacterium species.

Clause 21. The method of any one of Clauses 18-20, wherein the covalent organic framework is degraded in the subject after administration and delivery of the anti-mycobacterial agent.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. Moreover, any combination of the above-described elements in all possible variations thereof is encompassed by the invention unless otherwise indicated herein or otherwise clearly contradicted by context.

1. A covalent organic framework comprising dithioacetal linkages.

2. The covalent organic framework of claim 1, wherein the covalent organic framework comprises the reaction product of an aryl aldehyde and an aryl dithiol.

3. The covalent organic framework of claim 2, wherein the aryl aldehyde is an aryl dialdehyde having formula: 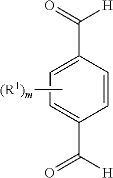
wherein in is 0, 1, or 2, and each R¹ is independently selected from —OH, —SH, —N₂, —N₃, halo, C₁-C₄ alkyl, C₂-C₄ alkenyl, C₂-C₄ alkynyl, C₁-C₄ alkoxy, and C₁-C₄ haloalkyl, wherein each R¹ can be optionally substituted with a reactive group.

4. The covalent organic framework of claim 2, wherein the aryl aldehyde is an aryl trialdehyde having formula: 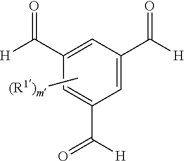
wherein m′ is 0, 1, 2, or 3, and each R^{1′} is independently selected from —OH, —SH, —N₂, —N₃, halo, C₁-C₄ alkyl, C₂-C₄ alkenyl, C₂-C₄ alkynyl, C₁-C₄ alkoxy, and C₁-C₄ haloalkyl, wherein each R^{1′} can be optionally substituted with a reactive group.

5. The covalent organic framework of claim 2, wherein the aryl dithiol has formula: 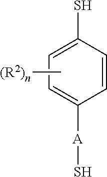
wherein:
n is 0, 1, or 2;
A is a bond or an aryl group substituted with 0, 1, or 2 R² groups; and
each R² is independently selected from —OH, —SH, —NH₂, —N₃, halo, C₁-C₄ alkyl, C₂-C₄ alkenyl, C₂-C₄ alkynyl, C₁-C₄ alkoxy, and C₁-C₄ haloalkyl, wherein each R⁷ can be optionally substituted with a reactive group.

6. The covalent organic framework of claim 1, wherein the covalent organic framework is further functionalized with one or more optionally substituted alkyl or heteroalkyl groups.

7. The covalent organic framework of claim 1, comprising the following structure: 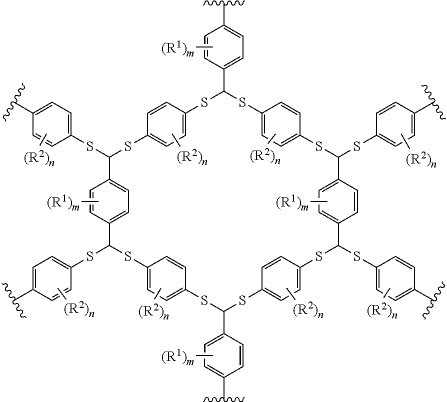
wherein:
each m is 0, 1, or 2;
each n is 0, 1, or 2,
each R¹ and R² is independently selected from —OH, —SH, —S—NO, —NH₂, —N₃, halo, C₁-C₄ alkyl, C₂-C₄ alkenyl, C₇-C₄ alkynyl, C₁-C₄ alkoxy, and C₁-C₄ haloalkyl, each of which can be optionally substituted with a reactive group.

8. The covalent organic framework of claim 1, comprising the following structure: 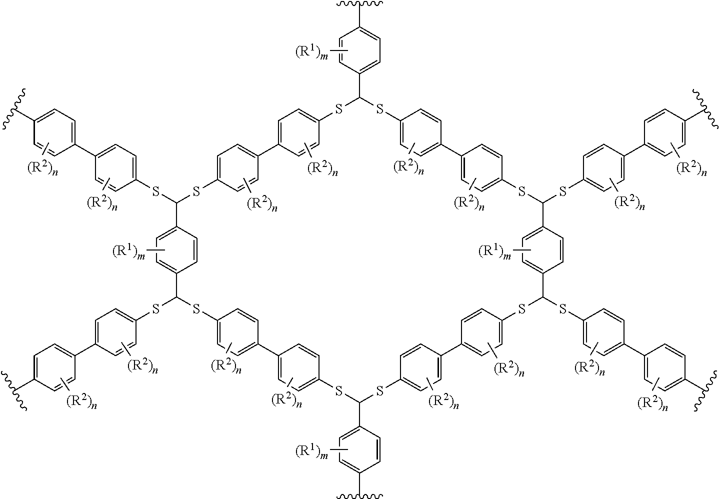
wherein:
each m is 0, 1, 2, 3, or 4;
each n is 0, 1, 2, 3, or 4;
each R¹ and R² is independently selected from —OH, —SH, —S—NO, —NH₂, —N₃, halo, C₁-C₄ alkyl, C₂-C₄ alkenyl, C₂-C₄ alkynyl, C₁-C₄ alkoxy, and C₁-C₄ haloalkyl, each of which can be optionally substituted with a reactive group.

9. The covalent organic framework of claim 1, wherein the covalent organic framework is further functionalized with one or more polyethylene glycol-containing groups.

10. The covalent organic framework of claim 1, wherein the covalent organic framework is loaded with an anti-mycobacterial agent.

11. The covalent organic framework of claim 10, wherein the anti-mycobacterial agent is isoniazid.

12. The covalent organic framework of claim 1, wherein the covalent organic framework is biodegradable.

13. A particle comprising the covalent organic framework of claim 1.

14. A method of synthesizing a covalent organic framework, comprising:
reacting an aryl aldehyde with an aryl dithiol.

15. The method of claim 14, wherein the aryl aldehyde is an aryl dialdehyde having formula: 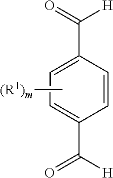
wherein m is 0, 1, 2, 3, or 4, and each R¹ is independently selected from —OH, —SH, —NH₂, —N₃, halo, C₁-C₄ alkyl, C₂-C₄ alkenyl. C₂-C₄ alkynyl, C₁-C₄ alkoxy, and C₁-C₄ haloalkyl, wherein R¹ can be optionally substituted with a reactive group.

16. The method of claim 14, wherein the aryl aldehyde is an aryl trialdehyde having formula: 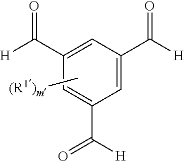
wherein m′ is 0, 1, 2, or 3, and each R^{1′} is independently selected from —OH, —SH, —NH₂, —N₃, halo, C₁-C₄ alkyl, C₂-C₄ alkenyl, C₂-C₄ alkynyl, C₁-C₄ alkoxy, and C₁-C₄ haloalkyl, wherein R^{1′} can be optionally substituted with a reactive group.

17. The method of claim 14, wherein the aryl dithiol has formula: 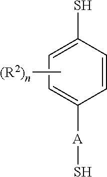
wherein:
n is 0, 1, 2, 3, or 4;
A is a bond or an aryl group substituted with 0, 1, or 2 R² groups; and
each R² is independently selected from —SH, —NH₂, —N₃, halo, C₁-C₄ alkyl, C₂-C₄ alkenyl, C₂-C₄ alkynyl, C₁-C₄ alkoxy, and C₁-C₄ haloalkyl, wherein R² can be optionally substituted with a reactive group.

18. A method of delivering an anti-mycobacterial agent to a subject in need thereof, comprising administering to the subject an effective amount of a covalent organic framework of claim 10.

19. The method of claim 18, wherein the administrating step comprises pulmonary administration.

20. The method of claim 18, wherein the subject is infected with a Mycobacterium species.

21. The method of claim 18, wherein the covalent organic framework is degraded in the subject after administration and delivery of the anti-mycobacterial agent.
